(19) United States
(12) Patent Application Publication
(10) Pub. No.: US 2014/0243625 A1
(43) Pub. Date: Aug. 28, 2014

(54) Coupling Apparatus and System for an Intravenous Fluid Delivery Tube and a Transcutaneous Sensor Cable

(71) Applicant: ivWatch, LLC, Williamsburg, VA (US)

(72) Inventor: Gary P. Warren, Williamsburg, VA (US)

(73) Assignee: ivWatch, LLC, Williamsburg, VA (US)

(21) Appl. No.: 14/192,059

(22) Filed: Feb. 27, 2014

Related U.S. Application Data
(60) Provisional application No. 61/770,641, filed on Feb. 28, 2013.

(51) Int. Cl. A61B 5/1459

(52) U.S. Cl. CPC A61B 5/1459
USPC 600/310

(57) ABSTRACT

A clip includes a first passage, a second passage, and a body. The first passage receives a tube that delivers an intravascular fluid in Animalia tissue. The second passage receives a cable of a sensor that aids in diagnosing at least one of infiltration and extravasation in the Animalia tissue. The body completely cinctures the first passage and includes a first cantilever arm that incompletely cinctures the second passage. A first arrangement of the body retains the cable in the second passage and a second arrangement of the body relinquishes the cable from the first arrangement.

### CROSS-REFERENCES TO RELATED APPLICATIONS

This application claims the priority of U.S. Provisional Application No. 61/770,641, filed 28 Feb. 2013, which is hereby incorporated by reference in its entirety.

### STATEMENT REGARDING FEDERALLY SPONSORED RESEARCH OR DEVELOPMENT

Not Applicable

### BACKGROUND OF THE INVENTION

FIGS. 13A and 13B show a typical arrangement for intravascular infusion. As the terminology is used herein, “intravascular” preferably refers to being situated in, occurring in, or being administered by entry into a blood vessel, thus “intravascular infusion” preferably refers to introducing a fluid or infusate into a blood vessel. Intravascular infusion accordingly encompasses both intravenous infusion (administering a fluid into a vein) and intra-arterial infusion (administering a fluid into an artery).

A cannula 20 is typically used for administering fluid via a subcutaneous blood vessel V. Typically, cannula 20 is inserted through skin S at a cannulation or cannula insertion site N and punctures the blood vessel V, for example, the cephalic vein, basilica vein, median cubital vein, or any suitable vein for an intravenous infusion. Similarly, any suitable artery may be used for an intra-arterial infusion.

Cannula 20 typically is in fluid communication with a fluid source 22. Typically, cannula 20 includes an extracorporeal connector, e.g., a hub 20a, and a transcutaneous sleeve 20b. Fluid source 22 typically includes one or more sterile containers that hold the fluid(s) to be administered. Examples of typical sterile containers include plastic bags, glass bottles or plastic bottles.

An administration set 30 typically provides a sterile conduit for fluid to flow from fluid source 22 to cannula 20. Typically, administration set 30 includes tubing 32, a drip chamber 34, a flow control device 36, and a cannula connector 38. Tubing 32 preferably extends along a longitudinal axis of administration set 30 from an inlet 30a to an outlet 30b and is typically made of polypropylene, nylon, or another flexible, strong and inert material. Preferably, inlet 30a is coupled at fluid source 22 and outlet 30b is coupled at hub 20a. Drip chamber 34 typically permits the fluid to flow one drop at a time for reducing air bubbles in the flow. Tubing 32 and drip chamber 34 are typically transparent or translucent to provide a visual indication of the flow. Typically, flow control device 36 is positioned upstream from drip chamber 34 for controlling fluid flow in tubing 32. Roller clamps and Dial-A-Flo®, manufactured by Hospira, Inc. (Lake Forest, Ill., US), are examples of typical flow control devices. Typically, cannula connector 38 and hub 20a provide a leak-proof coupling through which the fluid may flow. Luer-Lok™, manufactured by Becton, Dickinson and Company (Franklin Lakes, N.J., US), is an example of a typical leak-proof coupling.

Administration set 30 may also include at least one of a clamp 40, an injection port 42, a filter 44, an extension set 46, or other devices. Typically, clamp 40 pinches tubing 32 to cut-off fluid flow. Injection port 42 typically provides an access port for administering medicine or another fluid via cannula 20. Filter 44 typically purifies and/or treats the fluid flowing through administration set 30. For example, filter 44 may strain contaminants from the fluid. Typically, extension set 46 is a portion of tubing 32 that is coupled between cannula connector 38 and hub 20a. Extension 46 may have a reduced diameter bore for neonates or pediatric patients.

An infusion pump 50 may be coupled with administration set 30 for controlling the quantity or the rate of fluid flow to cannula 20. The Alaris® System manufactured by CareFusion Corporation (San Diego, Calif., US), BodyGuard® Infusion Pumps manufactured by CMA America, L.L.C. (Golden, Colo., US), and Flo-Gard® Volumetric Infusion Pumps manufactured by Baxter International Inc. (Deerfield, Ill., US) are examples of typical infusion pumps.

Intravenous infusion or therapy typically uses a fluid (e.g., infusate, whole blood, or blood product) to correct an electrolyte imbalance, to deliver a medication, or to elevate a fluid level. Typical infusates predominately consist of sterile water with electrolytes (e.g., sodium, potassium, or chloride), calories (e.g., dextrose or total parenteral nutrition), or medications (e.g., anti-infectives, anticonvulsants, antihyperuricemic agents, cardiovascular agents, central nervous system agents, chemotherapy drugs, coagulation modifiers, gastrointestinal agents, or respiratory agents). Examples of medications that are typically administered during intravenous therapy include acyclovir, allopurinol, amikacin, aminophylline, amiodarone, amphotericin B, ampicillin, carboplatin, cefazolin, cefotaxime, cefuroxime, ciprofloxacin, cisplatin, clindamycin, cyclophosphamide, diazepam, docetaxel, dopamine, doxorubicin, doxycycline, erythromycin, etoposide, fentanyl, fluorouracil, furosemide, ganciclovir, gemcitabine, gentamicin, heparin, imipenem, irinotecan, lorazepam, magnesium sulfate, meropenem, methotrexate, methylprednisolone, midazolam, morphine, nafcillin, ondansetron, paclitaxel, pentamidine, phenobarbital, phenytoin, piperacillin, promethazine, sodium bicarbonate, ticarcillin, tobramycin, topotecan, vancomycin, vinblastine and vincristine. Transfusions and other processes for donating and receiving whole blood or blood products (e.g., albumin and immunoglobulin) also typically use intravenous infusion.

Unintended infusing typically occurs when fluid from cannula 20 escapes from its intended vein/artery. Typically, unintended infusing causes an abnormal amount of the fluid to diffuse or accumulate in perivascular tissue P and may occur, for example, when (i) cannula 20 causes a vein/artery to rupture; (ii) cannula 20 improperly punctures the vein/artery; (iii) cannula 20 backs out of the vein/artery; (iv) cannula 20 is improperly sized; (v) infusion pump 50 administers fluid at an excessive flow rate; or (vi) the infusate increases permeability of the vein/artery. As the terminology is used herein, “tissue” preferably refers to an association of cells, intercellular material and/or interstitial compartments, and “perivascular tissue” preferably refers to cells, intercellular material and/or interstitial compartments that are in the general vicinity of a blood vessel and may become unintentionally infused with fluid from cannula 20. Unintended infusing by a non-vesicant fluid is typically referred to as “infiltration,” whereas unintended infusing by a vesicant fluid is typically referred to as “extravasation.”

The symptoms of infiltration or extravasation typically include blanching or discoloration of the skin S, edema, pain, or numbness. The consequences of infiltration or extravasation typically include skin reactions (e.g., blisters), nerve compression, compartment syndrome, or necrosis. Typical treatment for infiltration or extravasation includes applying warm or cold compresses, elevating an affected limb, administering hyaluronidase, phentolamine, sodium thiosulfate or dexrazoxane, fasciotomy, or amputation.

### BRIEF SUMMARY OF THE INVENTION

Embodiments according to the present invention are directed to a clip that includes a first passage, a second passage, and a body. The first passage is configured to receive a tube that delivers an intravascular fluid in Animalia tissue. The second passage is configured to receive a cable of a sensor that aids in diagnosing at least one of infiltration and extravasation in the Animalia tissue. The body completely cinctures the first passage and includes a first cantilever arm that incompletely cinctures the second passage. A first arrangement of the body is configured to retain the cable in the second passage and a second arrangement of the body is configured to release the cable from the first arrangement.

Other embodiments according to the present invention are directed to a clip that includes first and second surfaces, first and second passages, and a body. The second surface is spaced along a longitudinal axis from the first surface. The first passage is configured to receive a cable of a sensor that aids in diagnosing at least one of infiltration and extravasation in the Animalia tissue. The first passage extends along a first axis between the first and second faces. The second passage is configured to receive a tube that delivers an intravascular fluid in Animalia tissue. The second passage extends along a second axis between the first and second faces. The body is disposed between the first and second faces and separates the first and second passages.

Other embodiments according to the present invention are directed to a clip that includes first and second surfaces, first and second conjoined passages, and a body that is disposed between the first and second faces. The second surface is spaced along a longitudinal axis from the first surface. The first passage is configured to receive a cable of a sensor that aids in diagnosing at least one of infiltration and extravasation in the Animalia tissue. The first passage extends along a first axis between the first and second faces. The second passage is configured to receive a tube that delivers an intravascular fluid in Animalia tissue. The second passage extends along a second axis between the first and second faces.

Other embodiments according to the present invention are directed to a coupling system that includes an intravascular fluid delivery tube, a cable, and a clip set. The intravascular fluid delivery tube extends along a first axis between first and second ends. The cable extends along a second axis between a transcutaneous sensor and a connector. A first arrangement of the clip set is configured to couple the cable with respect to the intravascular fluid delivery tube. A second arrangement of the clip set is configured to decouple the cable from the first arrangement.

Other embodiments according to the present invention are directed to a system that includes a mono-directional carrier, a bi-directional carrier, and at least one clip. The mono-directional carrier extends along a first axis between an inlet and an outlet. The bi-directional carrier extends along a second axis between first and second ends. The at least one clip is configured in first and second arrangements. The first arrangement of the at least one clip is configured to couple the mono-directional carrier to the bi-directional carrier. The second arrangement of the at least one clip is configured to decouple at least one of the mono-directional and bi-directional carriers from the first arrangement.

### BRIEF DESCRIPTION OF THE DRAWINGS

The accompanying drawings, which are incorporated herein and constitute part of this specification, illustrate exemplary embodiments of the invention, and, together with the general description given above and the detailed description given below, serve to explain the features, principles, and methods of the invention.

FIG. 1 is a schematic view illustrating an electromagnetic radiation sensor shown contiguously engaging Animalia skin.

FIGS. 2A-2C are schematic cross-section views demonstrating how an anatomical change over time in perivascular tissue impacts the electromagnetic radiation sensor shown in FIG. 1.

FIG. 3 is a schematic view illustrating a coupling system according to the present disclosure. The system includes three clips for coupling an intravascular fluid delivery tube and a transcutaneous sensor cable.

FIG. 4 is a perspective view illustrating an individual clip of the coupling system shown in FIG. 3. Portions of the intravascular fluid delivery tube and transcutaneous sensor cable are schematically illustrated.

FIG. 5 is a schematic plan view illustrating another clip according to the present disclosure.

FIG. 6 is a schematic plan view illustrating another clip according to the present disclosure.

FIG. 7 is a schematic plan view illustrating another clip according to the present disclosure.

FIGS. 8 to 10 are schematic plan views illustrating three variations of another clip according to the present disclosure.

FIG. 11 is a schematic plan view illustrating another clip according to the present disclosure.

FIG. 12 is a schematic plan view illustrating another clip according to the present disclosure.

FIG. 13A is a schematic view illustrating a typical set-up for infusion administration.

FIG. 13B is a schematic view illustrating a subcutaneous detail of the set-up shown in FIG. 13A.

In the figures, the thickness and configuration of components may be exaggerated for clarity. The same reference numerals in different figures represent the same component.

### DETAILED DESCRIPTION OF THE INVENTION

The following description and drawings are illustrative and are not to be construed as limiting. Numerous specific details are described to provide a thorough understanding of the disclosure. However, in certain instances, well-known or conventional details are not described in order to avoid obscuring the description.

Reference in this specification to “one embodiment” or “an embodiment” means that a particular feature, structure, or characteristic described in connection with the embodiment is included in at least one embodiment according to the disclosure. The appearances of the phrases “one embodiment” or “other embodiments” in various places in the specification are not necessarily all referring to the same embodiment, nor are separate or alternative embodiments mutually exclusive of other embodiments. Moreover, various features are described that may be exhibited by some embodiments and not by others. Similarly, various features are described that may be included in some embodiments but not other embodiments.

The terms used in this specification generally have their ordinary meanings in the art, within the context of the disclosure, and in the specific context where each term is used. Certain terms in this specification may be used to provide additional guidance regarding the description of the disclosure. It will be appreciated that a feature may be described more than one-way.

Alternative language and synonyms may be used for any one or more of the terms discussed herein. No special significance is to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and is not intended to further limit the scope and meaning of the disclosure or of any exemplified term.

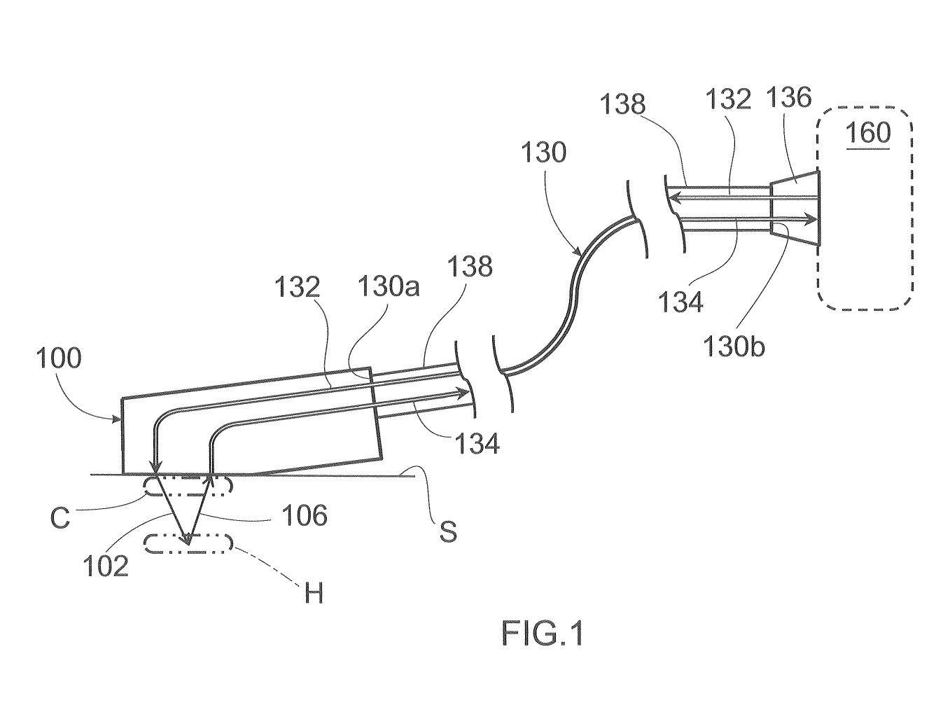

FIG. 1 shows an electromagnetic radiation sensor 100 that preferably includes an anatomic sensor. As the terminology is used herein, “anatomic” preferably refers to the structure of an Animalia body and an “anatomic sensor” preferably is concerned with sensing a change over time of the structure of the Animalia body. By comparison, a physiological sensor is concerned with sensing the functions or activities of an Animalia body, e.g., pulse or blood chemistry, at a point in time.

Electromagnetic radiation sensor 100 preferably is coupled with the skin S. Preferably, electromagnetic radiation sensor 100 is arranged to overlie a target area of the skin S. As the terminology is used herein, “target area” preferably refers to a portion of a patient's skin that is generally proximal to where an infusate is being administered and frequently proximal to the cannulation site N. Preferably, the target area overlies the perivascular tissue P. According to one embodiment, adhesion preferably is used to couple electromagnetic radiation sensor 100 to the skin S. According to other embodiments, any suitable coupling may be used that preferably minimizes relative movement between electromagnetic radiation sensor 100 and the skin S.

Electromagnetic radiation sensor 100 preferably emits and collects transcutaneous electromagnetic radiation signals, e.g., light signals. Preferably, electromagnetic radiation sensor 100 emits electromagnetic radiation 102 and collects electromagnetic radiation 106. Emitted electromagnetic radiation 102 preferably passes through the target area of the skin S toward the perivascular tissue P. Collected electromagnetic radiation 106 preferably includes a portion of emitted electromagnetic radiation 102 that is at least one of specularly reflected, diffusely reflected (e.g., due to elastic or inelastic scattering), fluoresced (e.g., due to endogenous or exogenous factors), or otherwise redirected from the perivascular tissue P before passing through the target area of the skin S.

The transcutaneous electromagnetic radiation signals emitted by electromagnetic radiation sensor 100 preferably are not harmful to an Animalia body. Preferably, the wavelength of emitted electromagnetic radiation 102 is longer than at least approximately 400 nanometers. The frequency of emitted electromagnetic radiation 102 therefore is no more than approximately 750 terahertz. According to one embodiment, emitted electromagnetic radiation 102 is in the visible radiation (light) or infrared radiation portions of the electromagnetic spectrum. Preferably, emitted electromagnetic radiation 102 is in the near infrared portion of the electromagnetic spectrum. As the terminology is used herein, “near infrared” preferably refers to electromagnetic radiation having wavelengths between approximately 600 nanometers and approximately 2,100 nanometers. These wavelengths correspond to a frequency range of approximately 500 terahertz to approximately 145 terahertz. A desirable range in the near infrared portion of the electromagnetic spectrum preferably includes wavelengths between approximately 800 nanometers and approximately 1,050 nanometers. These wavelengths correspond to a frequency range of approximately 375 terahertz to approximately 285 terahertz. According to other embodiments, electromagnetic radiation sensor 100 may emit electromagnetic radiation signals in shorter wavelength portions of the electromagnetic spectrum, e.g., ultraviolet light, X-rays or gamma rays, preferably when radiation intensity and/or signal duration are such that tissue harm is minimized.

Emitted and collected electromagnetic radiation 102 and 106 preferably share one or more wavelengths. According to one embodiment, emitted and collected electromagnetic radiation 102 and 106 preferably share a single peak wavelength, e.g., approximately 940 nanometers (approximately 320 terahertz). As the terminology is used herein, “peak wavelength” preferably refers to an interval of wavelengths including a spectral line of peak power. The interval preferably includes wavelengths having at least half of the peak power. Preferably, the wavelength interval is +/− approximately 20 nanometers with respect to the spectral line. According to other embodiments, emitted and collected electromagnetic radiation 102 and 106 preferably share a plurality of peak wavelengths, e.g., approximately 940 nanometers and approximately 650 nanometers (approximately 460 terahertz). According to other embodiments, a first one of emitted and collected electromagnetic radiation 102 and 106 preferably spans a first range of wavelengths, e.g., from approximately 600 nanometers to approximately 1000 nanometers. This wavelength range corresponds to a frequency range from approximately 500 terahertz to approximately 300 terahertz. A second one of emitted and collected electromagnetic radiation 102 and 106 preferably shares with the first range a single peak wavelength, a plurality of peak wavelengths, or a second range of wavelengths. Preferably, an optical power analysis at the wavelength(s) shared by emitted and collected electromagnetic radiation 102 and 106 provides an indication of anatomical change over time in the perivascular tissue P.

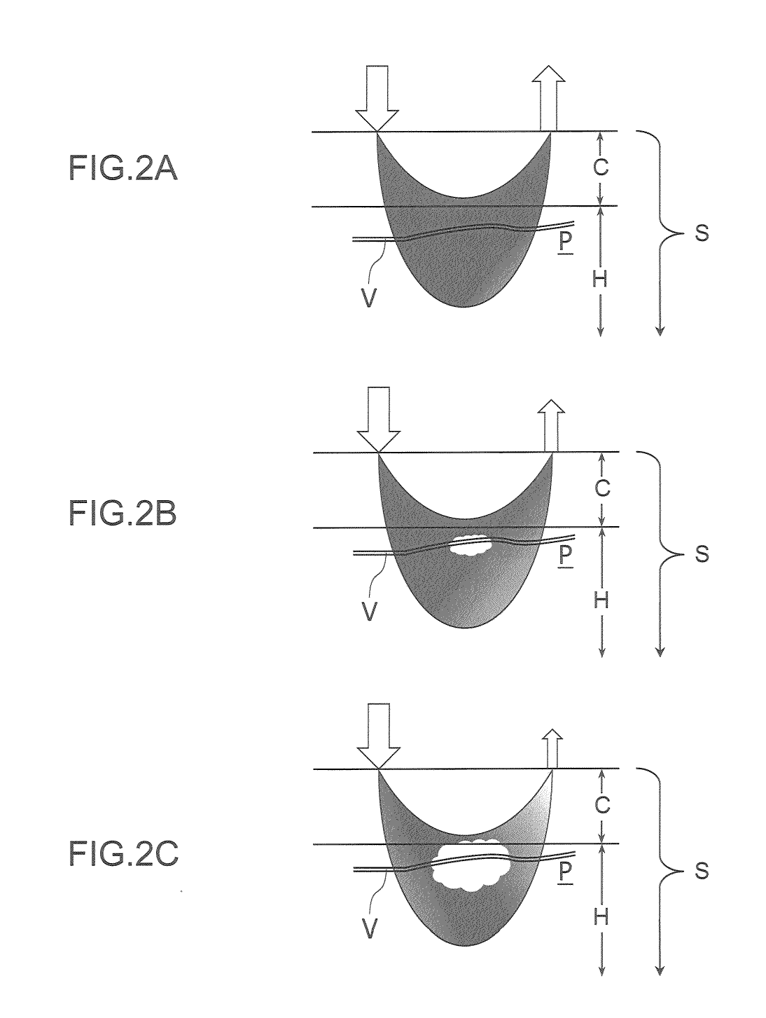

FIGS. 2A-2C schematically illustrate how an infiltration/extravasation event preferably evolves. FIG. 2A shows the skin S prior to an infiltration/extravasation event. Preferably, the skin S includes cutaneous tissue C, e.g., stratum corneum, epidermis and/or dermis, overlying subcutaneous tissue, e.g., hypodermis H. Blood vessels V suitable for intravenous therapy typically are disposed in the hypodermis H. FIG. 2B shows an infusate F beginning to accumulate in the perivascular tissue P. Accumulation of the infusate F typically begins in the hypodermis H, but may also begin in the cutaneous tissue C or at an interface of the hypodermis H with the cutaneous tissue C. FIG. 2C shows additional accumulation of the infusate F in the perivascular tissue P. Typically, the additional accumulation extends further in the hypodermis H but may also extend into the cutaneous tissue C. According to one embodiment, an infiltration/extravasation event generally originates and/or occurs in proximity to the blood vessel V, e.g., as illustrated in FIGS. 2A-2C. According to other embodiments, an infiltration/extravasation event may originate and/or occur some distance from the blood vessel V, e.g., if pulling on the cannula C or administration set 30 causes the cannula outlet to become displaced from the blood vessel V.

FIGS. 2A-2C also schematically illustrate the relative power of emitted and collected electromagnetic radiation 102 and 106. Preferably, emitted electromagnetic radiation 102 enters the skin S, electromagnetic radiation propagates through the skin S, and collected electromagnetic radiation 106 exits the skin S. Emitted electromagnetic radiation 102 is schematically illustrated with an arrow directed toward the skin S and collected electromagnetic radiation 106 is schematically illustrated with an arrow directed away from the skin S. Preferably, the relative sizes of the arrows correspond to the relative powers of emitted and collected electromagnetic radiation 102 and 106. The propagation is schematically illustrated with crescent shapes that preferably include the predominant electromagnetic radiation paths through the skin S from emitted electromagnetic radiation 102 to collected electromagnetic radiation 106. Stippling in the crescent shapes schematically illustrates a distribution of electromagnetic radiation power in the skin S with relatively lower power generally indicated with less dense stippling and relatively higher power generally indicated with denser stippling.

The power of collected electromagnetic radiation 106 preferably is impacted by the infusate F accumulating in the perivascular tissue P. Prior to the infiltration/extravasation event (FIG. 2A), the power of collected electromagnetic radiation 106 preferably is a fraction of the power of emitted electromagnetic radiation 102 due to electromagnetic radiation scattering and absorption by the skin S. Preferably, the power of collected electromagnetic radiation 106 changes with respect to emitted electromagnetic radiation 102 in response to the infusate F accumulating in the perivascular tissue P (FIGS. 2B and 2C). According to one embodiment, emitted and collected electromagnetic radiation 102 and 106 include near infrared electromagnetic radiation. The power of collected electromagnetic radiation 106 preferably decreases due to scattering and/or absorption of near infrared electromagnetic radiation by the infusate F. The compositions of most infusates typically are dominated by water. Typically, water has different absorption and scattering coefficients as compared to the perivascular tissue P, which contains relatively strong near infrared energy absorbers, e.g., blood. At wavelengths shorter than approximately 700 nanometers (approximately 430 terahertz), absorption coefficient changes preferably dominate due to absorption peaks of blood. Preferably, scattering coefficient changes have a stronger influence than absorption coefficient changes for wavelengths between approximately 800 nanometers (approximately 375 terahertz) and approximately 1,300 nanometers (approximately 230 terahertz). In particular, propagation of near infrared electromagnetic radiation in this range preferably is dominated by scattering rather than absorption because scattering coefficients have a larger magnitude than absorption coefficients. Absorption coefficient changes preferably dominate between approximately 1,300 nanometers and approximately 1,500 nanometers (approximately 200 terahertz) due to absorption peaks of water. Therefore, the scattering and/or absorption impact of the infusate F accumulating in the perivascular tissue P preferably is a drop in the power signal of collected electromagnetic radiation 106 relative to emitted electromagnetic radiation 102. According to other embodiments, a rise in the power signal of collected electromagnetic radiation 106 relative to emitted electromagnetic radiation 102 preferably is related to infusates with different scattering and absorption coefficients accumulating in the perivascular tissue P. Thus, the inventors discovered, inter alia, that fluid changes in perivascular tissue P over time, e.g., due to an infiltration/extravasation event, preferably are indicated by a change in the power signal of collected electromagnetic radiation 106 with respect to emitted electromagnetic radiation 102.

Electromagnetic radiation sensor 100 preferably aids healthcare givers in identifying infiltration/extravasation events. Preferably, changes in the power signal of collected electromagnetic radiation 106 with respect to emitted electromagnetic radiation 102 alert a healthcare giver to perform an infiltration/extravasation evaluation. The evaluation that healthcare givers perform to identify infiltration/extravasation events typically includes palpitating the skin S in the vicinity of the target area, observing the skin S in the vicinity of the target area, and/or comparing limbs that include and do not include the target area of the skin S.

Electromagnetic radiation sensor 100 preferably communicates via a cable 130 with an electro-optical unit 160. Preferably, electro-optical unit 160 supplies emitted electromagnetic radiation 102 to electromagnetic radiation sensor 100 over a first transmission line 132 and receives collected electromagnetic radiation 106 from electromagnetic radiation sensor 100 over a second transmission line 134. According to one embodiment, first and second transmission lines 132 and 134 preferably include respective sets of optical fibers. Preferably, emitted electromagnetic radiation 102 includes a first near-infrared wavelength signal that is transmitted by first transmission line 132 over at least one optical fiber, and collected electromagnetic radiation 106 includes a second near-infrared wavelength signal that is transmitted by second transmission line 134 over at least one optical fiber. Accordingly, an advantage of this embodiment preferably is that electromagnetic radiation sensor 100 and cable 130 substantially exclude ferrous materials that may interact with magnetic fields, e.g., a magnetic field induced during a magnetic resonance imaging procedure. According to another embodiment, first and second transmission lines 132 and 134 preferably include respective sets of metal wires. Preferably, emitted electromagnetic radiation 102 includes a first electrical signal that is transmitted by first transmission line 132 over at least one metal wire to a light source, e.g., a light emitting diode, disposed in electromagnetic radiation sensor 100, and collected electromagnetic radiation 106 includes a second electrical signal that is transmitted by second transmission line 134 over at least one metal wire from a light collector, e.g., a photodiode, disposed in electromagnetic radiation sensor 100. Accordingly, an advantage of this embodiment preferably is that cable 130 and electromagnetic radiation sensor 100 preferably are disposable after a single-use because metal wires typically are less expensive than optical fibers.

Cable 130 preferably extends along a longitudinal axis between first and second ends 130a and 130b. According to one embodiment, electromagnetic radiation sensor 100 preferably is hermetically coupled at first end 130a. Preferably, a connector 136 is hermetically coupled at second end 130b to facilitate repeatable coupling and decoupling of cable 130 with respect to a mating feature of electro-optical unit 106. According to other embodiments, first end 130a preferably includes a connector to facilitate repeatable coupling and decoupling of cable 130 with respect to a mating feature of electromagnetic radiation sensor 100.

First and second transmission lines 132 and 134 preferably are bundled in cable 130. Preferably, a sheath 138 cincturing first and second transmission lines 132 and 134 extends between first and second ends 130a and 130b. Sheath 138 preferably includes a thermoplastic urethane or another suitably flexible material. Tecoflex®, manufactured by The Lubrizol Corporation (Wickliffe, Ohio, USA), is an example of a typical thermoplastic urethane for bundling optical fibers in a sheath. According to one embodiment, sheath 138 preferably includes an inner core contiguously engaging first and second transmission lines 132 and 134, and a relatively thin (e.g., approximately 0.010 inches) outer core that further includes an antimicrobial additive, such as a quaternary ammonium. Biosafe®, manufactured by BIOSAFE Inc. (Pittsburgh, Pa., USA), is an example of a typical antimicrobial additive. Preferably, the inner and outer layers of sheath 138 are co-extruded around first and second transmission lines 132 and 134. According to other embodiments, a biocompatible material preferably is incorporated throughout sheath 138. According to other embodiments, sheath 138 preferably includes electromagnetic radiation shielding. According to other embodiments, sheath 138 preferably includes a formation that resists crushing and/or breaking first and second transmission lines 132 and 134. According to other embodiments, one or both of first and second transmission lines 132 and 134 preferably include individual encasements that are cinctured by sheath 138.

The inventors discovered a problem regarding dislodging cannula 20 or electromagnetic radiation sensor 100 due to individually snagging or pulling administration set 30 or cable 130. The inventors also discovered, inter alia, that mutually controlling administration set 30 and cable 130 reduces opportunities for dislodging cannula 20 or electromagnetic radiation sensor 100.

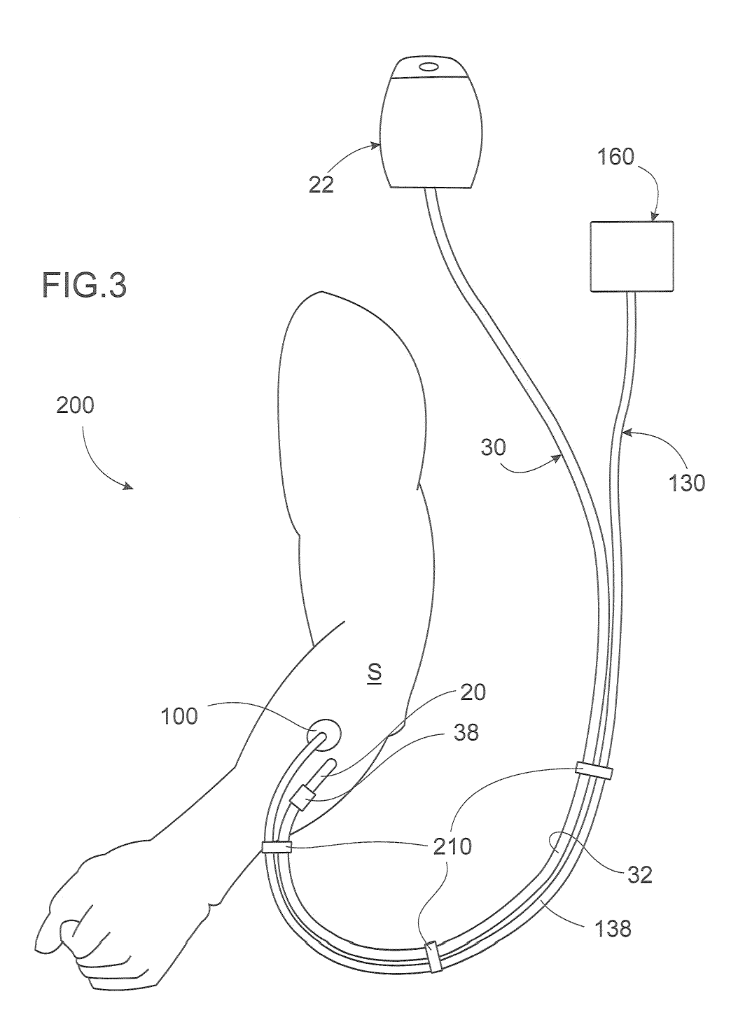

FIG. 3 schematically illustrates a system 200 that preferably couples and decouples an intravascular fluid delivery tube, e.g., administration set 30, and a transcutaneous sensor cable, e.g., cable 130. Preferably, coupling system 200 includes at least one clip 210 disposed along the longitudinal axes of administration set 30 and cable 130. FIG. 3 shows three clips 210; however, the number of clips 210 may be greater than three or less than three. Preferably, several clips 210 are disposed at intervals along the longitudinal axes of administration set 30 and cable 130. According to one embodiment, a first clip 210 preferably is disposed near the cannula connector 38 at a location where tubing 32 is formed in a bight typically described as a “Hoop.” According to other embodiments, clips 210 preferably slide along the longitudinal axes of administration set 30. The locations and intervals of individual clips 210 are therefore preferably variable along the longitudinal axes of administration set 30. According to other embodiments, clips 210 preferably are fixed along the longitudinal axes of administration set 30. According to other embodiments, a combination of fixed and slideable clips 210 is disposed along the longitudinal axes of administration set 30.

Individual clips 210 preferably have first and second arrangements. According to the first arrangement, clip 210 preferably contiguously engages tubing 32 and sheath 138 to retain a longitudinal axis 30c of administration set 30 proximate to a longitudinal axis 130c of cable 130. According to the second arrangement, clip 210 preferably relinquishes at least one of the longitudinal axes 30c and 130c from the first arrangement. Thus, the inventors discovered, inter alia, that a clip 210 for mutually controlling administration set 30 and cable 130 preferably reduces opportunities for dislodging cannula 20 or electromagnetic radiation sensor 100.

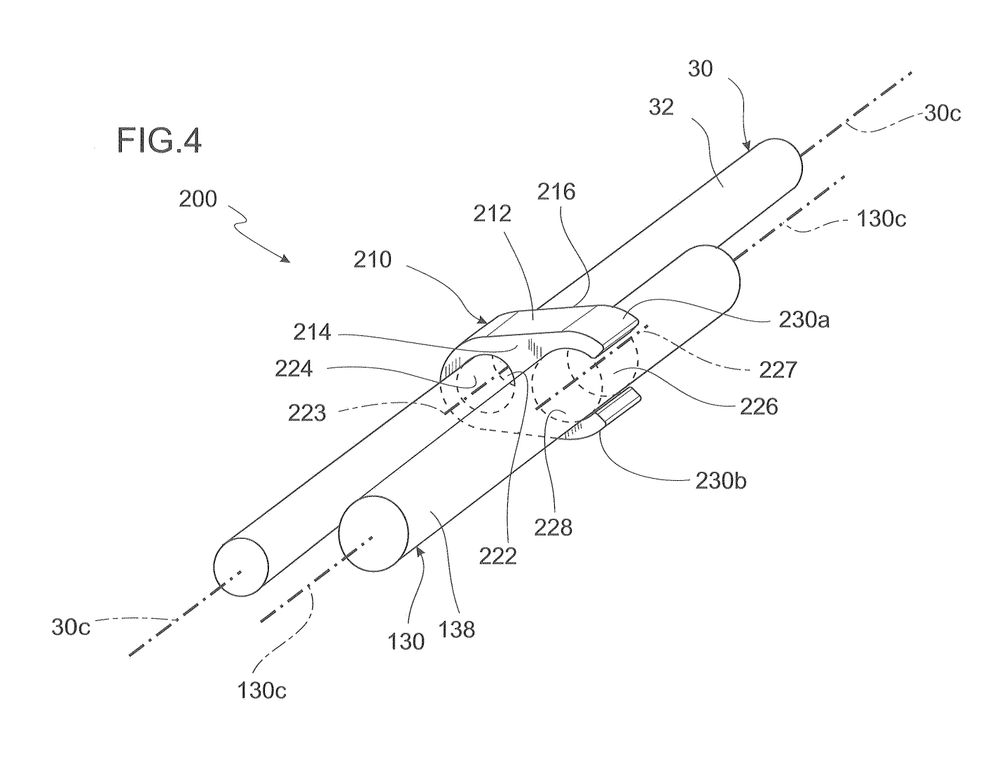

FIG. 4 illustrates portions of administration set 30 and cable 130 coupled in accordance with the first arrangement of clip 210. Preferably, clip 210 includes a body 212 disposed between a first surface 214 that is longitudinally spaced from a second surface 216. First and second passages 222 and 226 preferably extend through body 212 between first and second surfaces 214 and 216. Preferably, body 212 includes first and second walls 224 and 228 that couple first and second surfaces 214 and 216. First and second walls 224 and 228 preferably at least partially define first and second passages 222 and 226, respectively. According to one embodiment, the first arrangement of clip 210 includes tubing 32 of administration set 30 being received in first passage 222 and sheath 138 of cable 130 being received in second passage 226. According to other embodiments, tubing 32 is received in first passage 222 sheath 138 is received in second passage 226.

The first arrangement of clip 210 preferably mutually controls administration set 30 and cable 130 by retaining longitudinal axes 30 and 130c in relative proximity to one another. First and second passages 222 and 226 preferably extend longitudinally through body 212 along axes 223 and 227, which generally coincide with longitudinal axes 30c and 130c, respectively, in the first arrangement of clip 210. Preferably, axes 223 and 227 are generally centered in passages 222 and 226, respectively. According to the embodiment shown in FIG. 4, body 212 separates passages 222 and 226, wall 224 completely cinctures axis 223, and wall 228 incompletely cinctures axis 227. Preferably, wall 228 incompletely cinctures axis 227 because of a longitudinally extending slot through which cable 130 may be laterally inserted into or extracted from passage 226. Wall 224 preferably requires administration set 30 to be longitudinally inserted, e.g., along axis 223. Accordingly, the second arrangement of clip 210 preferably includes relinquishing cable 130 from passage 226 by laterally displacing longitudinal axis 130c through the slot in wall 228.

Body 212 preferably includes one or more cantilever arms that are resiliently deformable between the first and second arrangements of clip 210. According to the embodiment shown in FIG. 4, first and second cantilever arms 230a and 230b preferably project from body 212. Preferably, wall 228 includes portions of body 212, first cantilever arm 230a, and second cantilever arm 230b. First and second cantilever arms 230a and 230b preferably are resiliently deformable in a generally radial direction with respect to axis 227. Preferably, transitions between the first and second arrangements of clip 210 include resiliently deforming first and second cantilever arms 230a and 230b so as to sufficiently enlarge the arcuate slot in wall 228 such that cable 130 may be laterally displaced in/out of passage 228. First and second cantilever arms 230a and 230b preferably resume a generally nominal position while retaining cable 130 in passage 228 (first arrangement of clip 210, as shown in FIG. 4) and after relinquishing cable 130 from passage 228 (second arrangement of clip 210).

The cross-sectional area of passage 222 may be smaller, generally equivalent, or larger than the cross-sectional area of passage 226. Preferably, the cross-sectional areas of passages 222 and 226, which are measured orthogonal to first and second axes 223 and 227, generally correspond to the cross-sectional areas of tubing 32 and sheath 138, respectively. According to the embodiment shown in FIG. 4, the cross-sectional area of passages 222 is less than the cross-sectional area of passage 226. According to other embodiments, if the cross-sectional area of tubing 32 is greater than the cross-sectional area of sheath 138, then the cross-sectional area of passage 222 preferably is greater than the cross-sectional area of passage 226. According to other embodiments, if the cross-sectional areas of tubing 32 and sheath 138 are generally similar, then the cross-sectional areas of passages 222 and 226 are preferably generally similar. According to other embodiments, the cross-sectional area of a passage may be larger than the corresponding cross-sectional area of administration set 30 or cable 130 that is received in the passage. Accordingly, clip 210 preferably is slideable along the longitudinal axis 30c or 130c with respect to tubing 32 or sheath 138, respectively.

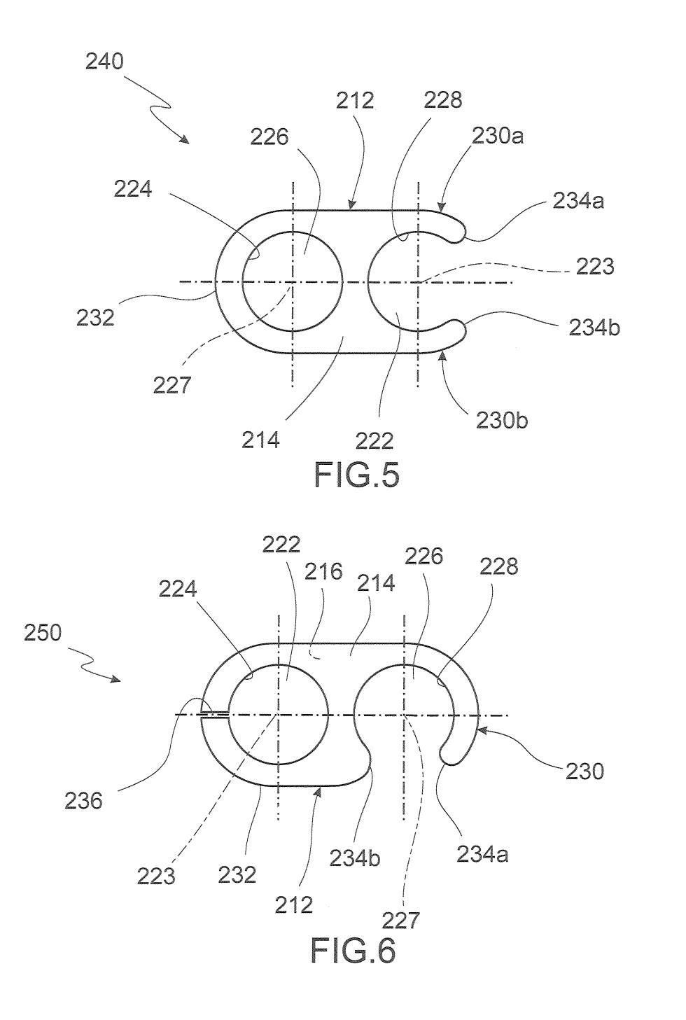

FIG. 5 is a schematic plan view illustrating a clip 240 according to the present disclosure. As compared to clip 210 (FIG. 4), wall 228 incompletely cinctures a passage 222 receiving an intravascular fluid delivery tube, wall 224 completely cinctures a passage 226 receiving a transcutaneous sensor cable, and the cross-sectional areas of passages 222 and 226 are generally equivalent. Preferably, the intravascular fluid delivery tube is laterally inserted into or extracted from passage 222 and the transcutaneous sensor cable is longitudinally inserted along axis 227 in passage 226. Accordingly, the second arrangement of clip 240 preferably includes relinquishing the intravascular fluid delivery tube from passage 222 by laterally displacement of axis 223 with respect to axis 227.

FIG. 5 also shows interior and exterior walls of clip 240. The interior walls of clip 240 preferably include walls 224 and 228. Preferably, an exterior wall 232 of clip 240 is coupled with interior wall 228 at a pair of longitudinal seams 234a and 234b disposed at the tips of cantilever arms 230a and 230b, respectively. Interior wall 224 preferably completely cinctures axis 227 and therefore is not coupled with exterior wall 232.

FIG. 6 is a schematic plan view illustrating a clip 250 according to the present disclosure. As compared to clips 210 (FIG. 4) and 240 (FIG. 5), body 212 is relatively thin (e.g., the longitudinally spacing between first and second surfaces 214 and 216 is relatively small), wall 228 incompletely cinctures passage 227 and includes a portion of body 212 and portion of a single cantilever arm 230, and a narrow gap 236 couples interior wall 224 and exterior wall 232. Interior wall 228 preferably is coupled with exterior wall 232 at longitudinal seams 234a and 234b. Preferably, longitudinal seam 234a is disposed at the tip of cantilever arm 230 and longitudinal seam 234b is disposed on body 212.

Accessing passage 222 preferably requires a relatively greater effort as compared to accessing passage 226. A method of accessing passage 222 preferably includes (i) applying a first longitudinally directed force to first surface 214; and (ii) applying a second longitudinally directed force to second surface 216. Accordingly, the first and second forces are directed in generally opposite longitudinal directions and preferably are applied proximate to opposite sides of narrow gap 236. Preferably, body 212 is therefore resiliently deformed into a generally helical form about axis 223 so as to longitudinally separate edges of narrow gap 236 and thereby increase accessibility to passage 222. Preferably, clip 250 provides a number of advantages including (i) providing an option for nurses or other practitioners to laterally access passage 222 in addition to the option for longitudinally sliding along axis 223; and (ii) requiring an increased effort by pediatric or curious patients to separate clip 250 from coupling system 200.

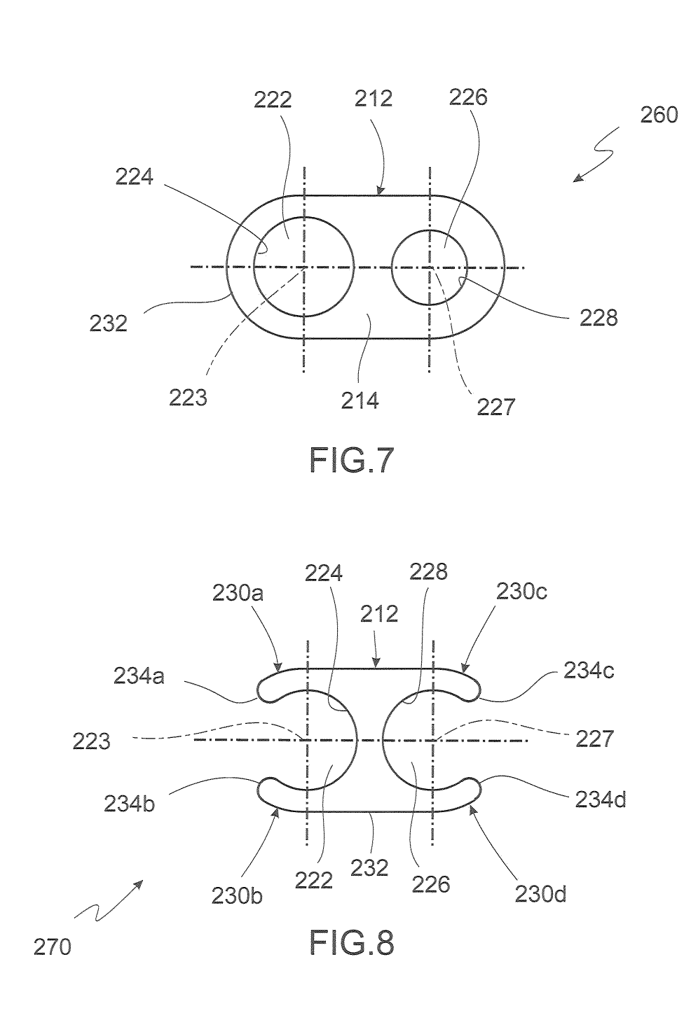

FIG. 7 is a schematic plan view illustrating a clip 260 according to the present disclosure. As compared to clips 210 (FIG. 4), 240 (FIG. 5) and 250 (FIG. 6), wall 224 completely cinctures passage 222 receiving an intravascular fluid delivery tube, wall 228 completely cinctures a passage 226 receiving a transcutaneous sensor cable, and the cross-sectional area of passage 222 is greater than the cross-sectional area of passage 226. Preferably, the intravascular fluid delivery tube is longitudinally inserted along axis 223 in passage 222 and the transcutaneous sensor cable is longitudinally inserted along axis 227 in passage 226. Accordingly, the second arrangement of clip 240 preferably includes relinquishing at least one of the intravascular fluid delivery tube and transcutaneous sensor cable by longitudinal displacement along axis 223 or axis 227, respectively. The interior walls of clip 260 include walls 224 and 228, which completely cincture axes 223 and 227, respectively, and therefore preferably are not coupled with exterior wall 232 of clip 236.

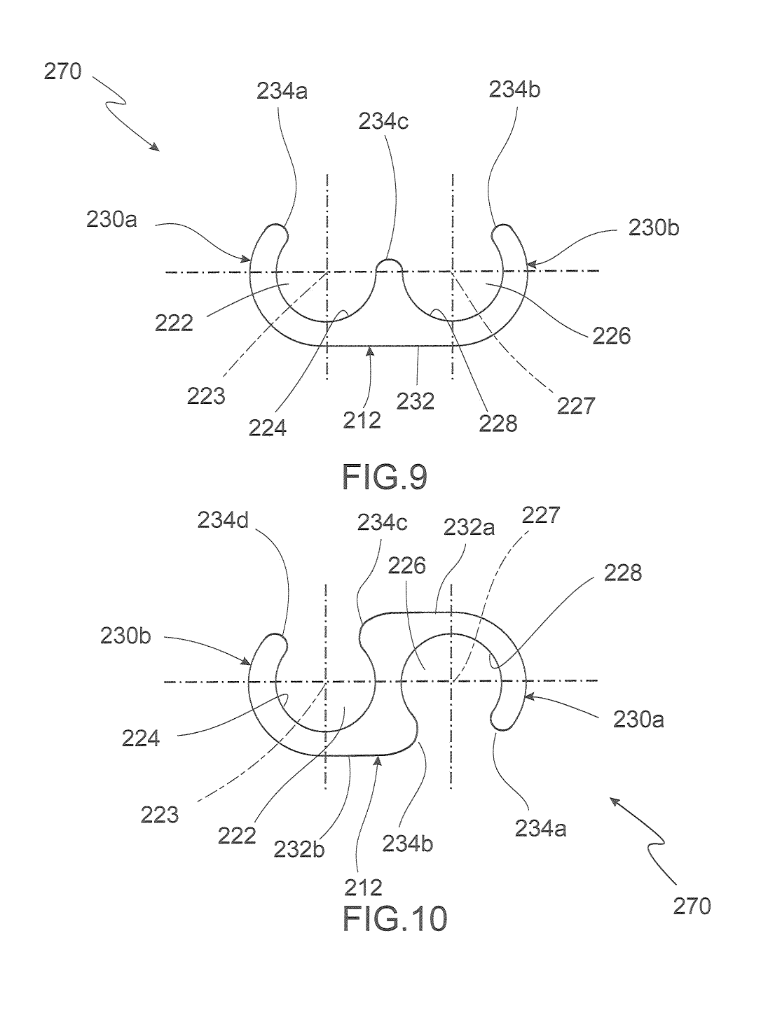

FIGS. 8 to 10 are schematic plan views illustrating three variations of a clip 270 according to the present disclosure. As compared to clips 210 (FIG. 4), 240 (FIG. 5), 250 (FIG. 6) and 260 (FIG. 7), walls 224 and 228 preferably incompletely cincture passages 222 and 226, respectively. As shown in FIG. 8, clip 270 preferably includes four cantilever arms 230a-230d. Preferably, interior wall 224 includes portions of first cantilever arm 230a, body 212 and second cantilever arm 230b, and interior wall 228 includes portions of third cantilever arm 230c, body 212 and fourth cantilever arm 230d. Exterior wall 232 is preferably coupled with interior walls 224 and 228 at four longitudinal seams 234a-234d disposed at the tips of cantilever arms 230a-230d, respectively. As shown in FIG. 9, clip 270 preferably includes first and second cantilever arms 230a and 230b. Preferably, interior wall 224 includes portions of first cantilever arm 230a and body 212, and interior wall 228 includes portions of body 212 and second cantilever arm 230b. Exterior wall 232 is preferably coupled with interior walls 224 and 228 at two longitudinal seams 234a and 234b disposed at the tips of first and second cantilever arms 230a and 230b, respectively. Preferably, interior walls 224 and 228 are coupled together at a third longitudinal seam 234c. As shown in FIG. 10, clip 270 preferably includes first and second cantilever arms 230a and 230b. Preferably, interior wall 224 includes portions of first cantilever arm 230b and body 212, and interior wall 228 includes portions of body 212 and second cantilever arm 230a. Two exterior walls 232a and 232b are preferably coupled with interior walls 224 and 228 at four longitudinal seams 234a-234d. Preferably, first longitudinal seam 234a is disposed at the tip of cantilever arm 230a, second and third longitudinal seams 234b and 234c are disposed on body 212, and fourth longitudinal seam 234d is disposed at the tip of cantilever arm 230b.

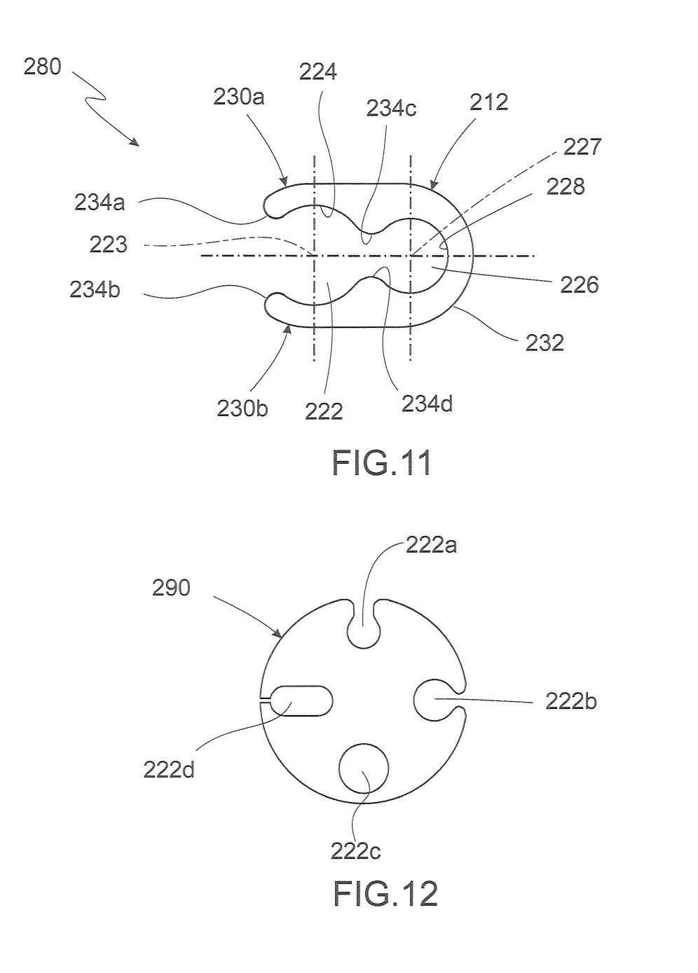

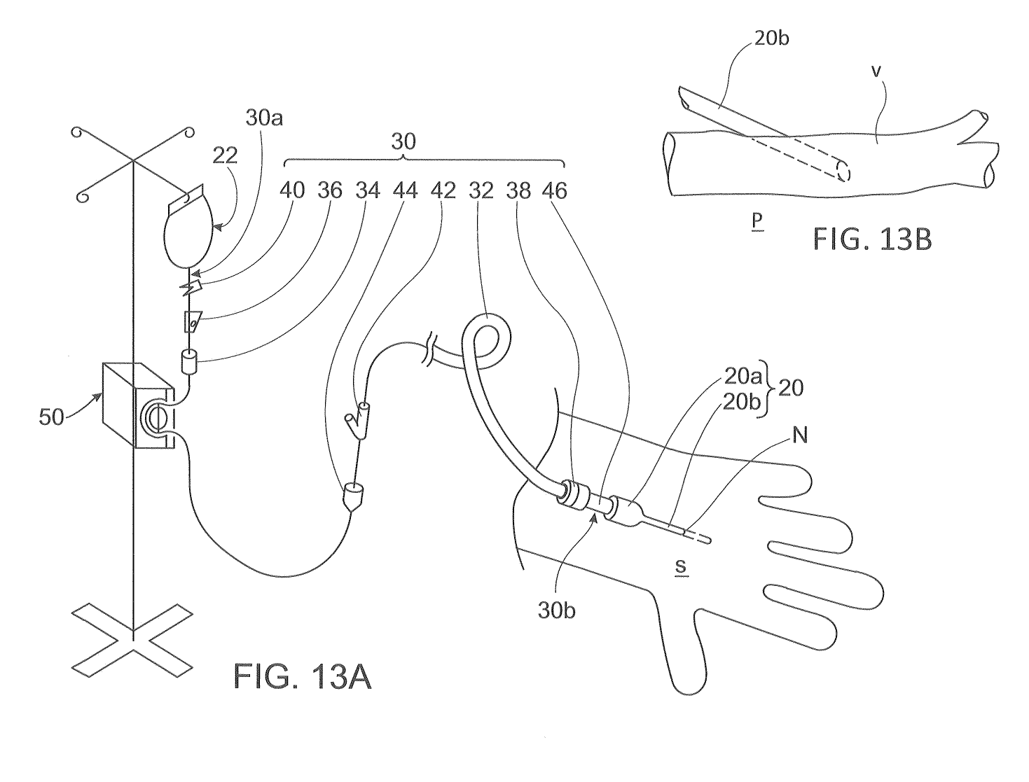

FIG. 11 is a schematic plan view illustrating a clip 280 according to the present disclosure. As compared to clips 210 (FIG. 4), 240 (FIG. 5), 250 (FIG. 6), 260 (FIG. 7) and 270 (FIGS. 8 to 10), walls 224 and 228 are conjoined. Clip 280 preferably includes first and second cantilever arms 230a and 230b. Preferably, body 212 includes interior wall 224 and portions of first and second cantilever arms 230a and 230b include interior wall 228. Exterior wall 232 is preferably coupled with interior wall 224 at two longitudinal seams 234a and 234b disposed at the tips of first and second cantilever arms 230a and 230b, respectively. Preferably, interior walls 224 and 228 are coupled together at two longitudinal seams 234c and 234d. As shown in FIG. 11, wall 224 incompletely cinctures passage 222 receiving an intravascular fluid delivery tube and wall 228 incompletely cinctures a passage 226 receiving a transcutaneous sensor cable. Preferably, the first arrangement of clip 280 includes laterally inserting the transcutaneous sensor cable through passage 222 into passage 226 and subsequently laterally inserting the intravascular fluid delivery tube into passage 222. The second arrangement of clip 240 preferably includes relinquishing the intravascular fluid delivery tube from passage 222 by laterally displacement with respect to axis 223. Preferably, the cross-sectional area of passage 222 is greater than the cross-sectional area of passage 226.

FIG. 12 is a schematic plan view illustrating a clip 290 according to the present disclosure. Preferably, clip 290 includes a plurality of passages (four passages 222a-222d are indicated on FIG. 12) for receiving and mutually controlling at least two lines. Examples of lines preferably include one or more intravascular fluid delivery tubes, transcutaneous sensor cables, physiological monitor cables, or combinations thereof. Preferably, the size, shape, type, and location on clip 290 of individual passages 222a-222d may be similar, different, or combinations thereof.

The inventors therefore discovered, inter alia, that a coupling system to mutually control a mono-directional carrier and a bi-directional carrier preferably includes at least one clip having first and second passages. Preferably, a first arrangement of an individual clip preferably includes the mono-directional carrier, e.g., an intravascular fluid delivery tubing line, being coupled in the first passage and the bi-directional carrier, e.g., emitter and detector lines for a transcutaneous anatomical sensor, being coupled in the second passage. Preferably, a second arrangement of the individual clip decouples at least one of the mono-directional and bi-directional carriers from the first arrangement.

While the present invention has been disclosed with reference to certain embodiments, numerous modifications, alterations, and changes to the described embodiments are possible without departing from the sphere and scope of the present invention, as defined in the appended claims. Accordingly, it is intended that the present invention not be limited to the described embodiments, but that it has the full scope defined by the language of the following claims, and equivalents thereof.

What is claimed is:

1. A clip comprising:
a first passage configured to receive a tube delivering an intravascular fluid in Animalia tissue;
a second passage configured to receive a cable of a sensor aiding in diagnosing at least one of infiltration and extravasation in the Animalia tissue; and
a body completely cincturing the first passage and including a first cantilever arm incompletely cincturing the second passage, the body including
a first arrangement configured to retain the cable in the second passage; and
a second arrangement configured to release the cable from the first arrangement.

2. The clip of claim 1 wherein the first cantilever arm extends between a first base and a first tip.

3. The clip of claim 2 wherein the first base is resiliently coupled to the body.

4. The clip of claim 2 wherein the first tip bends toward the body.

5. The clip of claim 1 wherein the body includes a second cantilever arm.

6. The clip of claim 5 wherein the second cantilever arm extends between a second base and a second tip.

7. The clip of claim 6 wherein the second base is resiliently coupled to the body.

8. The clip of claim 6 wherein the second tip bends toward the body.

9. The clip of claim 5 wherein the first and second cantilever arms are configured to be biased into contiguous engagement with generally opposite sides of the cable in the first arrangement.

10. The clip of claim 5 wherein the first and second cantilever arms are configured to be displaced apart in the second arrangement.

11. A clip comprising:
a first surface;
a second surface spaced along a longitudinal axis from the first surface;
a first passage configured to receive a cable of a sensor aiding in diagnosing at least one of infiltration and extravasation in the Animalia tissue, the first passage extending along a first axis between the first and second faces;
a second passage configured to receive a tube delivering an intravascular fluid in Animalia tissue, the second passage extending along a second axis between the first and second faces;
a body disposed between the first and second faces and separating the first and second passages.

12. The clip of claim 11 wherein the first and second axes are generally parallel to the longitudinal axis.

13. The clip of claim 11 wherein the body comprises first and second walls coupling the first and second faces, the first wall at least partially defines the first passage and, and the second wall at least partially defines the second passage.

14. The clip of claim 13 wherein the first wall incompletely cinctures the first axis and the second wall completely cinctures the second axis.

15. The clip of claim 13 wherein the first wall completely cinctures the first axis and the second wall incompletely cinctures the second axis.

16. The clip of claim 13 wherein the first wall completely cinctures the first axis and the second wall completely cinctures the second axis.

17. The clip of claim 13 wherein the first wall incompletely cinctures the first axis and the second wall incompletely cinctures the second axis.

18. The clip of claim 11 wherein the first passage has a first cross-sectional area perpendicular to the first axis and the second passage has a second cross-sectional area perpendicular to the second axis.

19. The clip of claim 18 wherein the first cross-sectional area is greater than the second cross-sectional area.

20. The clip of claim 18 wherein the first cross-sectional area is less than the second cross-sectional area.

21. The clip of claim 18 wherein the first and second cross-sectional areas are approximately equal.

22. The clip of claim 11 wherein the body comprises at least one exterior wall and at least one interior wall, the exterior and interior walls couple the first and second faces, and the at least one interior wall at least partially defines the first and second passages.

23. The clip of claim 22 wherein the at least one interior wall and a single exterior wall are coupled at two seams.

24. The clip of claim 22 wherein a pair of interior walls and a pair of exterior walls are coupled at four seams.

25. The clip of claim 22 wherein a pair of interior walls are coupled at one seam and the pair of interior walls are coupled at two seams with a single exterior wall.

26. The clip of claim 11, comprising a third passage extending along a third axis between the first and second faces, wherein the body separates the first, second and third passages.

27. A clip comprising:
a first surface;
a second surface spaced along a longitudinal axis from the first surface;
a first passage configured to receive a cable of a sensor aiding in diagnosing at least one of infiltration and extravasation in the Animalia tissue, the first passage extending between the first and second faces;
a second passage configured to receive a tube delivering an intravascular fluid in Animalia tissue, the second passage extending between the first and second faces;
a body disposed between the first and second faces;
wherein the first and second passages are conjoined.

28. The clip of claim 27 wherein the first and second axes are generally parallel to the longitudinal axis.

29. The clip of claim 27 wherein the first and second passages are defined by a wall coupling the first and second faces, the wall being disposed on opposite sides of the first and second axes.

30. The clip of claim 29 wherein the first wall incompletely cinctures the first axis and the second wall incompletely cinctures the second axis.

31. The clip of claim 27 wherein the body comprises an exterior wall and an interior wall, the exterior and interior walls couple the first and second faces, the interior wall partially defines the first and second passages, and the interior and exterior walls are coupled at two seams.

32. The clip of claim 31 wherein the interior wall comprises first and second portions, the first portion partially defines the first passage, and the second portion partially defines the second passage.

33. The clip of claim 32 wherein the interior wall comprises a ridge between the first and second portions.

34. A coupling system comprising:
an intravascular fluid delivery tube extending along a first axis between first and second ends;
a cable extending along a second axis between a transcutaneous sensor and a connector; and
a clip set including
a first arrangement of the clip set being configured to couple the cable with respect to the intravascular fluid delivery tube; and
a second arrangement of the clip set being configured to decouple the cable from the first arrangement.

35. The coupling system of claim 34 wherein the first end is configured to receive an infusate, and the second end comprises a cannula.

36. The coupling system of claim 35 wherein the cannula is configured to penetrate Animalia tissue at an insertion site and intravascularly deliver an intravenous fluid.

37. The coupling system of claim 34 wherein the transcutaneous sensor is configured to aid in diagnosing at least one of infiltration and extravasation in perivascular Animalia tissue.

38. The coupling system of claim 34 wherein the clip set is configured to retain the first axis generally parallel to the second axis.

39. The coupling system of claim 38 wherein the clip set comprises a plurality of clips, and individual clips are disposed at intervals along the first axis.

40. The coupling system of claim 39 wherein individual clips contiguously engage the cable at intervals along the second axis in the first arrangement.

41. The coupling system of claim 34 wherein individual clips of the clip set include—
a first passage configured to receive the intravascular fluid delivery tube;
a second passage configured to receive the cable in the first arrangement; and
a body defining the first and second passages.

42. The coupling system of claim 41 wherein the body comprises first and second walls, the first wall completely cincturing the first passage, and the second wall incompletely cincturing the second passage.

43. The coupling system of claim 42 wherein the first wall is symmetrically disposed about the first axis in the first arrangement and the second wall is symmetrically disposed about the second axis in the first arrangement.

44. The coupling system of claim 41 wherein the body comprises a first cantilever arm incompletely cincturing the second passage, the first cantilever arm extending between a first base and a first tip, and the first base being resiliently coupled to the body.

45. The coupling system of claim 44 wherein the body comprises a second cantilever arm incompletely cincturing the second passage, the second cantilever arm extending between a second base and a second tip, and the second base being resiliently coupled to the body.

46. The coupling system of claim 45 wherein the first and second cantilever arms are configured to be biased into contiguous engagement with generally opposite sides of the cable in the first arrangement, and the first and second cantilever arms are configured to be displaced apart in the second arrangement.

47. A system comprising:
a mono-directional carrier extending along a first axis between an inlet and an outlet;
a bi-directional carrier extending along a second axis between first and second ends; and
at least one clip being configured in first and second arrangements, the first arrangement coupling the mono-directional carrier to the bi-directional carrier, and the second arrangement decoupling at least one of the mono-directional and bi-directional carriers from the first arrangement.

48. The system of claim 47 wherein the mono-directional carrier is configured to intravenously deliver an infusate, and the bi-directional carrier is configured to aid in diagnosing perivascular infiltration by the infusate.

49. The system of claim 47 wherein each individual clip includes—
a first passage configured to receive the mono-directional carrier;
a second passage configured to receive the bi-directional carrier in the first arrangement; and
a body defining the first and second passages.

50. The system of claim 47 wherein a plurality of clips are disposed at intervals along the first and second axes.

51. The system of claim 47 wherein the mono-directional carrier comprises an administration set configured to carry an infusate from the inlet to the outlet.

52. The system of claim 51 wherein the administration set comprises an infusate source, a cannula and tubing, the infusate supply is disposed at the inlet, the cannula is disposed at the outlet, and the tubing carries the infusate from the infusate supply to the cannula.

53. The system of claim 47 wherein the bi-directional carrier comprises a first and second sets of optical fibers, the first set of optical fibers is configured to carry a first optical signal from the first end to the second end, and the second set of optical fibers is configured to carry a second optical signal from the second end to the first end.

54. The system of claim 53 wherein wavelengths of the first and second optical signals are between approximately 600 nanometers and approximately 1,800 nanometers.

55. The system of claim 53 wherein wavelengths of the first and second optical signals are centered about approximately 940 nanometers.
